(19) United States
(12) Patent Application Publication
(10) Pub. No.: US 2018/0045700 A1
(43) Pub. Date: Feb. 15, 2018

(54) Perishable Food Detection System

(71) Applicant: Wal-Mart Stores, Inc., Bentonville, AR (US)

(72) Inventors: Matthew Dwain Biermann, Fayetteville, AR (US); Steven Jackson Lewis, Bentonville, AR (US); Nicholaus Adam Jones, Fayetteville, AR (US)

(21) Appl. No.: 15/653,786

(22) Filed: Jul. 19, 2017

Related U.S. Application Data
(60) Provisional application No. 62/375,015, filed on Aug. 15, 2016.

(51) Int. Cl. G01N 33/02; G06K 19/07; G08B 21/24; G01N 27/22

(52) U.S. Cl. CPC G01N 33/02; G01N 27/223; G06K 19/0717; G08B 21/24

(57) ABSTRACT

Described in detail herein are methods and systems for detecting moisture dissipated from perishable foods. Perishable foods can be disposed in shelving units and moisture sensors can be disposed with respects to the to the perishable foods. The moisture sensor can detect moisture dissipated by the perishable foods within range of the moisture sensor. The moisture sensors can transmit the amount of moisture detected to the computing system. The computing system can determine whether at least one perishable food item within the range of the moisture sensors is damaged or decomposing based on the amount of moisture detected.

### CROSS-REFERENCE TO RELATED PATENT APPLICATIONS

This application claims priority to U.S. Provisional Application No. 62/375,015 filed on Aug. 15, 2016, the content of which is hereby incorporated by reference in its entirety.

### BACKGROUND

Damaged or decomposing perishable foods stored within containers can go undetected, which can lead to odors, contamination of other physical objects in proximity to the decomposing physical objects, and/or other undesirable results.

### BRIEF DESCRIPTION OF DRAWINGS

Illustrative embodiments are shown by way of example in the accompanying drawings and should not be considered as a limitation of the present disclosure:

FIG. 1A is a block diagram of an exemplary shelving unit disposed in a facility implementing the moisture detecting system according to the present disclosure;

FIG. 1B is a block diagram of an exemplary shelving unit disposed in a facility implementing the moisture detecting system according to the present disclosure;

FIG. 2 illustrates an exemplary network environment of a computing system in accordance with exemplary embodiments of the present disclosure;

FIG. 3 illustrates an exemplary network environment of a computing system in accordance with exemplary embodiments of the present disclosure; and

FIG. 4 is a flowchart illustrating a perishable food detection system according to exemplary embodiments of the present disclosure.

### DETAILED DESCRIPTION

Described in detail herein are methods and systems for detecting damaged or decomposing perishable foods. For example, perishable foods can be disposed in shelving units. Embodiments of the shelving units can be a refrigerated to maintain the perishable foods at a temperature that is lower than the ambient temperature of the environment surrounding the shelving units. Some of the perishable foods can be damaged and/or decomposing and should be removed from the shelving units. Embodiments of the present disclosure can detect damaged or decomposing perishable foods using moisture sensors disposed with respects to the to the perishable foods that can detect moisture being dissipated by the damaged or decomposing perishable foods. The moisture sensors can output a signal corresponding to an amount of moisture detected to a computing system, which can determine whether at least one perishable food item within the range of at least one of the moisture sensors is damaged or decomposing based on the amount of moisture detected by one or more of the moisture sensors.

In exemplary embodiments, the perishable food monitoring system can include the shelving unit including a support surface for supporting physical objects, first set of moisture sensors disposed above and spaced away from the support surface, and a computing system. For example, the first set of sensors can be formed as an array that is disposed parallel to the support surface. The first set of moisture sensors can be configured to detect moisture being dissipated by the physical objects supported by the support surface and to output electrical signals corresponding to detected moisture being dissipated by the physical objects.

The computing system can be operatively coupled to the first set of the moisture sensors, and can be programmed to receive a (first) electrical signal from at least one of the moisture sensors included in the first set. Based on the electrical signal received by the computing system, the computing system can determine a level of moisture being dissipated by at least a subset of the physical objects and determine whether at least one of the physical objects in the subset is damaged or decomposing in response to a determination that the level of moisture exceeds a threshold moisture value. The computing system can estimate a location of the at least one damaged or decomposing physical object in response to determining the level of moisture exceeds the threshold moisture value and determining a first sensor location for the at least one sensor that detected the excess moisture. In response to estimating the location at which the damaged or decomposing physical object is disposed, the computing system can transmit an alert using one or more modes of communication over one or more communication channels.

In accordance with embodiments of the present disclosure, the computing system can be programmed to determine the level of moisture being dissipated by the at least subset of the physical objects decreases based on the first electrical signal received from the at least one sensor and determine that the at least one of the physical object in the subset which is determined to be damaged or decomposing is removed from the shelving unit in response to the decrease.

In accordance with embodiments of the present disclosure, the computing system can be programmed to determine the level of moisture being dissipated by at least the subset of the physical objects increases subsequent to the decrease based on the first electrical signal received from the at least one sensor and determine the at least one of the physical objects in the subset which is determined to be damaged or decomposing is placed back on the shelving unit. The computing system can dynamically change the threshold moisture value based on a change detected in one or more environmental parameters by at least two sensors in the first set and/or in response to not detecting at least one of the physical objects in the subset is damaged or decomposing. In some embodiments, one of the environmental parameters can be humidity.

Embodiments of the present disclosure can include a second set of sensors disposed in an array perpendicular to the support surface. The computing system can be programmed to receive a second electrical signal from at least one sensor in the second set of sensors and determine whether the at least one of the physical objects in the subset of physical objects is damaged or decomposing based on the second electrical signal. The computing system can be programmed to estimate the location of the at least one damaged or decomposing physical object producing excess moisture by determining the first sensor location of the at least one sensor from the first set of sensors and a second sensor location of the at least one sensor from the second set of sensors that detect the excess moisture.

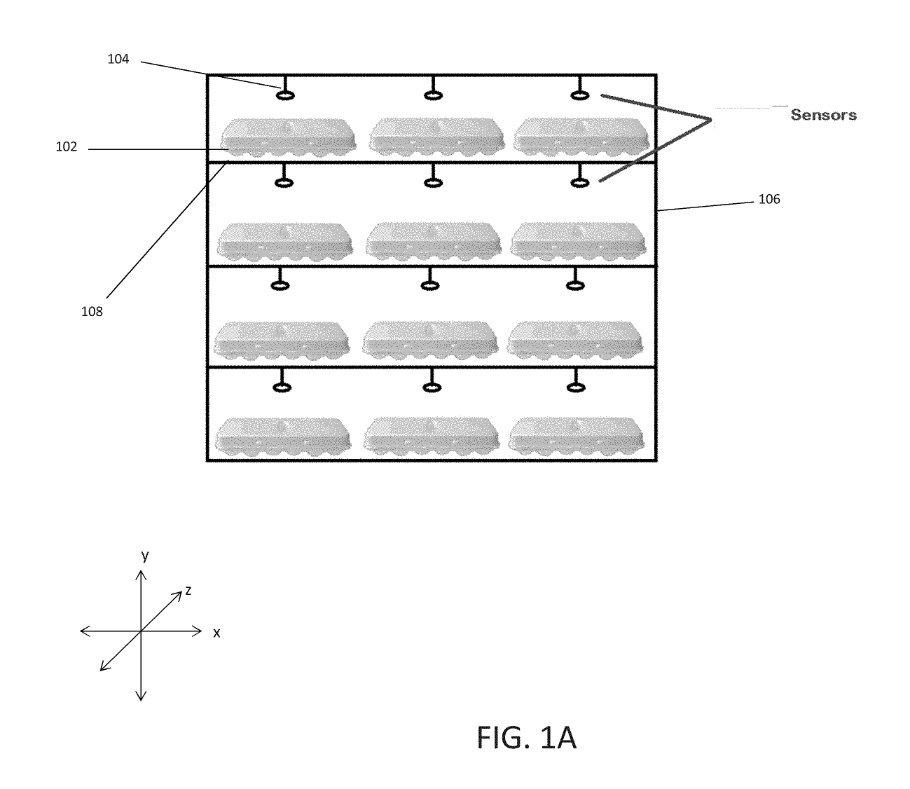

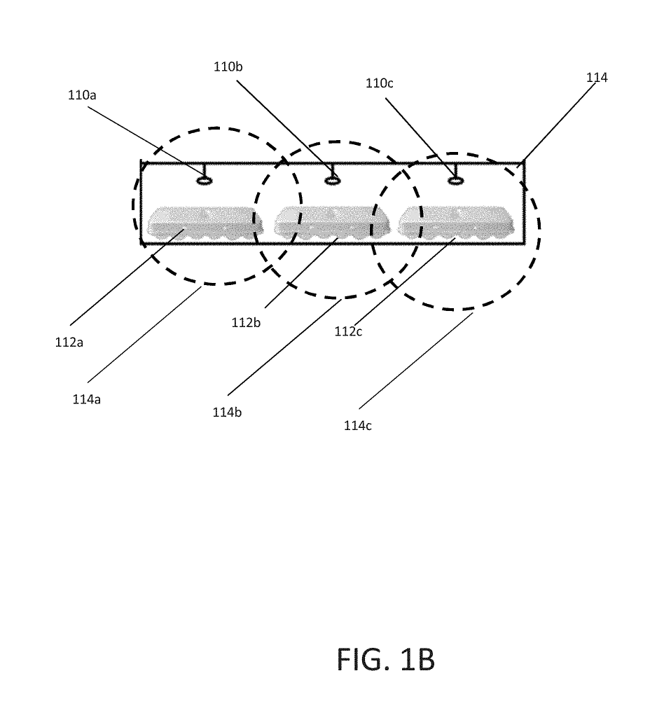

FIG. 1A is a block diagram of an exemplary shelving unit 106 disposed in a facility according to the present disclosure. The shelving unit 106 can hold, store and support sets of physical objects 102, which can be supported by support surfaces 108. The physical objects 102 can be edible or perishable products, which dissipate increased levels of moisture when damaged or decomposing The moisture sensors 104 disposed above and spaced away from the support surfaces 108, and can be configured to detect moisture being dissipated by the physical objects 102 supported by the support surfaces 108. The moisture sensors 104 can output electrical signals corresponding to detected moisture being dissipated in their proximity (e.g., moisture being produced by the physical objects 102 and/or moisture generally in environment). The moisture sensors 104 can be disposed in arrays parallel to the supporting surfaces 108 (e.g., along an x-axis). Each array can be disposed above one of the supporting surfaces 108 and each moisture sensor in each array can be positioned to be generally aligned over a physical object, a set of physical objects, or a stack of physical objects as described herein. In some embodiments, the moisture sensors 104 can be disposed in arrays extending perpendicularly with respect to the supporting surfaces 108 of the shelving unit 106 (e.g., along a y-axis). In some embodiments, a grid of moisture sensors 104 can be formed the arrays of moisture sensors extending parallel to the support surfaces and the arrays of moisture sensors extending perpendicularly to the supporting surfaces. The moisture sensors 104 can detect moisture within an predetermined range in an environment and/or can have specified sensitivity. At least one physical object 102 can be within the predetermined range of each moisture sensor 104 and the respective moisture sensor can detect the moisture dissipated by the physical object within the range of the moisture sensor. In some embodiments, moisture dissipated by at least one physical object 102 can be detected by at least a subset of the moisture sensors 104 over time. For example, one of the moisture sensors 104 that is closest in proximity to the at least one physical object 102, which is dissipating moisture, can detect the moisture first in time, the next closest one of the moisture sensors 104 can detect the moisture second in time, and one of the moisture sensors 104 that is farthest away from the at least one physical object 104 can detect the moisture last in time. It can be appreciated the moisture sensors 104 can be embodied as infrared sensors, olfactometers, oxygen sensor, carbon dioxide sensors, electrochemical gas sensors, electronic noses, hydrogen sensors, hydrogen sulfide sensors, microwave chemistry sensor, humistors, gas detectors, dew warning sensors, hygrometer, electrochemical gas sensors, air flow meters, gas meters, water meters, and any other type of sensors configured to detect damaged or decomposing physical objects 102.

In some embodiments, the physical objects 102 can form rows and columns in the shelving unit 106. At least one moisture sensor 104 can be disposed along each row of physical objects 102 and/or at least one moisture sensor 104 can be disposed over each column of the physical objects 102. In some embodiments, the moisture sensors 104 can be disposed along each row and column.

FIG. 1B is a block diagram of an exemplary shelving unit 114 disposed in a facility implementing a moisture detecting system according to the present disclosure. The shelving unit 114 can hold, store and support physical objects 112a-c. Moisture sensors 110a-c can be disposed along the shelf of the shelving unit, positioned above the physical objects 112a-c.

Each of the moisture sensors 110a-c can detect dissipation of moisture within a predetermined range and/or with a specified sensitivity. For example, moisture sensor 110a can generally detect moisture in a range 114a, the moisture sensor 110b can generally detect moisture in a range 114b and the moisture sensor 110c can generally detect moisture in a range 114c. The range 114a can include physical object 112a entirely and partially physical object 112b. The range 114b can include physical object 112b entirely and partially physical objects 112a and 112c. The range 114c can include physical object 112c entirely and partially physical object 112b. Therefore, the moisture sensors 110a-c can detect when the physical object(s) in their range (either partially or entirely) are dissipating moisture. In response to detecting moisture, the moisture sensors 110a-c can generate electrical signals corresponding to an amount of moisture detected. The moisture sensors 110a-c can transmit the electrical signal to a computing system.

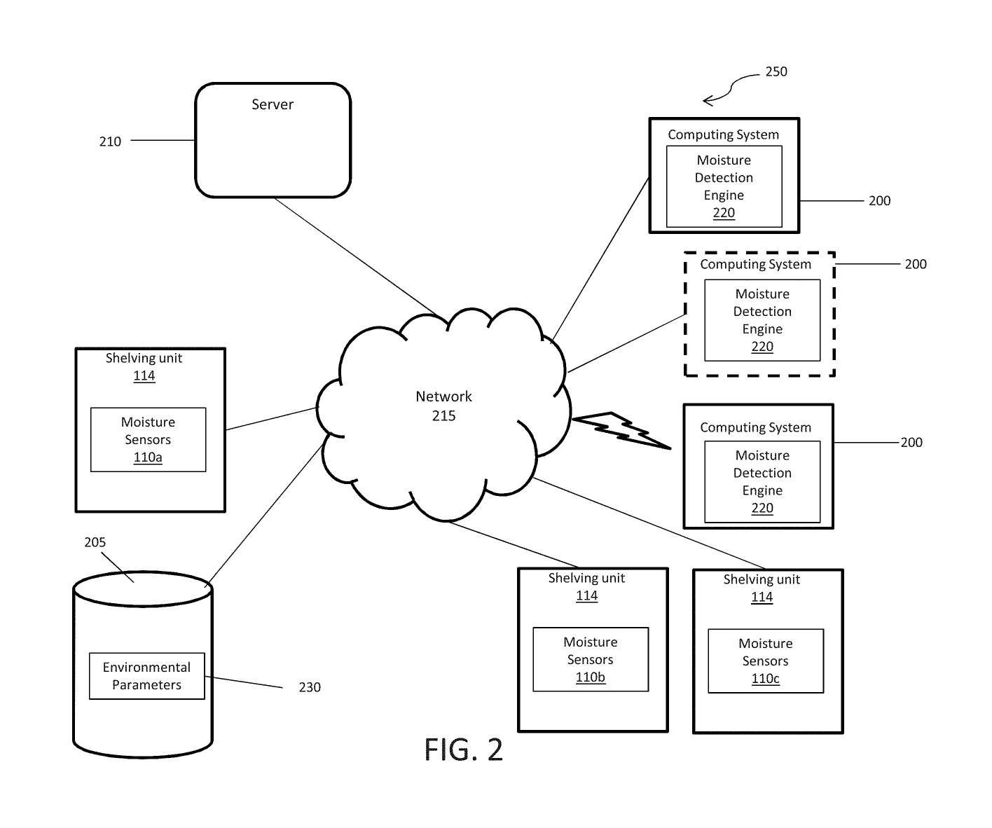

FIG. 2 illustrates detection perishable food monitoring system 250 according to exemplary embodiments. The system 250 can include one or more databases 205, one or more servers 210, one or more computing systems 200, and shelving units 114 with moisture sensors 110a-c as described herein. In exemplary embodiments, the computing system 200 is in communication with the databases 205, a server 210, and moisture sensors 110a-c, via a communications network 215. The computing system 200 can implement at least one instance of moisture detection engine 220.

In an example embodiment, one or more portions of the communications network 215 can be an ad hoc network, an intranet, an extranet, a virtual private network (VPN), a local area network (LAN), a wireless LAN (WLAN), a wide area network (WAN), a wireless wide area network (WWAN), a metropolitan area network (MAN), a portion of the Internet, a portion of the Public Switched Telephone Network (PSTN), a cellular telephone network, a wireless network, a WiFi network, a WiMax network, any other type of network, or a combination of two or more such networks.

The server 210 and the databases 205 are connected to the communications network 215 via a wired connection. Alternatively, the server 210 and the databases 205 can be connected to the network 215 via a wireless connection. The server 210 includes one or more computers or processors configured to communicate with the computing system 200 and the databases 205, via the network 215. The server 210 hosts one or more applications configured to interact with one or more components computing system 200 and/or facilitates access to the content of the databases 205. The databases 205 may store information/data, as described herein. For example, the databases 205 can include an environmental parameters database 230 that stores sensed or measured environmental parameters of the shelving units 106, environmental parameters from other locations or from other parts of the facility (e.g., from sensors that are located remotely from the shelving units 106), and/or the environment surrounding the shelving units 106. The environment parameters can impact the level of moisture in around the shelving units 106. The databases 205 and server 210 can be located at one or more geographically distributed locations from each other or from the computing system 200. Alternatively, the databases 205 can be included within server 210.

In exemplary embodiments, the computing system 200 can receive electrical signals from the moisture sensors 110a-c indicating moisture detected by the moisture sensors 110a-c disposed in the shelving unit 114 and can generate a baseline moisture level. The baseline moisture level can be stored in the environmental parameters database 230. Physical objects can be disposed on the shelving unit and a first one of the moisture sensors 110a-c can detect moisture being dissipated from a subset of physical objects on the shelving unit. The first moisture sensor can be disposed above the set of physical objects and can output a first electrical signal corresponding to a level of moisture detected by the first one of the moisture sensors 110a-c. The first electrical signal can be received by the computing system 200, which execute the moisture detection engine 220 to determine whether the first electrical signal indicates a change in moisture level being sensed by the first one of the moisture sensors 110a-c. For example, the computing system 200 can monitor the first electrical signal over time and compare a value of the first electrical signal at an earlier time to a value of the first electrical signal being received contemporaneously by the computing system 200 to detect a change in the moisture level being sensed by the moisture sensors 110a-c. The change in the moisture level can correspond to an increased level of moisture being dissipated by the subset of physical objects. The computing system 200 can execute the moisture detection engine 220 to determine a level of moisture being contemporaneously sensed by one or more of the other moisture sensors in the shelving unit 114 and can query the environmental parameters database 230 to retrieve the baseline moisture level. The computing system 200 can eliminate the amount of moisture caused by environmental parameters affecting the moisture in the shelving unit 114. For example, if the computing system 200, executing the moisture detection engine 220, determines that the level of moisture sensed by each of the moisture sensors 110a-c increases by a substantially equal or similar amount (e.g., with a specified percentage), the increase can be factored out of the change in the level of moisture detected by the first one of the moisture sensors 110a-c and a determination of whether damaged or decomposing physical objects are present. The computing system 200 can dynamically determine a threshold moisture level to be exceed before the computing system 200 determines, via the moisture detection engine 220, that a damaged or decomposing physical object is present. For example, the computing system 200 can use the current moisture sensed by each of the moisture sensors 110a-c and/or one or more stored environmental parameters to set the threshold value. The computing system 200 can execute the moisture detection engine 220 to compare the moisture level determined based on the first electrical signal and the threshold moisture level of the location of the first sensor and determine whether the moisture level determined based on the first electrical signal exceeds the threshold moisture level of the location of the first moisture sensor. In response to determining the moisture level determined from the first electrical signal exceeds the threshold moisture level of the location of the first moisture sensor the computing system 200 can execute the moisture detection engine 220 to determine at least one physical object within the range of the first moisture sensor is damaged or decomposing. The computing system 200 can estimate a location of the damaged or decomposing physical object based on the location of the first moisture sensor that sensed the excess moisture. The computing system 200 can execute the detection engine to transmit an alert in response to determining the physical objects and determining the location of the damaged and decomposing physical object in the set of physical objects.

In some embodiments, a second change in the moisture levels can be detected by the first moisture sensor within a specified time period after the first change. The computing system 200 can compare the moisture levels determined based on the first and second changes in the moisture levels. In response to determining the moisture levels corresponding to the second change are less than the moisture levels corresponding to the first change, the computing system 200 can execute the detection engine to determine that a damaged or decomposing physical object has been removed from the shelving unit.

In some embodiments, a third change in the moisture levels can be detected by the first moisture sensor within a specified time period after the second change. The computing system 200 can compare the moisture levels corresponding the second and third changes. In response to determining the moisture levels of the third change exceed the moisture levels of the second change, the computing system 200 can execute the detection engine determine the damaged or decomposing physical object was placed back on the shelving unit and to transmit an alert. The alert can include instructions to manually confirm the physical object is damaged or decomposing.

In some embodiments, a set moisture sensors 110a-c including a second moisture sensor can be disposed perpendicular to a supporting surface upon which the subset of physical objects are disposed as described herein. The computing system 200 can execute the detection engine to receive electrical signals from the first and second moisture sensor along with the locations of the first and second sensor. The computing system 200 can determine, via the detection engine, that the at least one physical object is damaged or decomposing from the subset of physical objects within the range of the first moisture sensor and within the range of the second moisture sensor.

In some embodiments, the computing system 200 can receive electrical signals from two or more sensors and the location of the sensors (e.g., the first and second moisture sensors). The computing system 200 can determine the change in moisture based on the electrical signals received from the two more sensors. The computing system 200 can determine a change in the environmental parameters in the locations of the two or more sensors. The computing system 200 can dynamically update the environmental parameters database 230 with the change in the environmental parameters for the locations of the two or more sensors. The computing system 200 can dynamically change the threshold based on the change in the environmental parameters.

In some embodiments, the shelving units with the moisture sensors 110a-c can be refrigerated units that maintain the temperature of the physical object at a temperature that is below an ambient temperature of the environment surrounding the shelving unit. The environmental parameter can be but are not limited to, increased and decreased humidity, increased and decreased temperature, condensation, liquid contained within the physical objects and other environmental parameters causing moisture.

As a non-limiting example, the perishable food monitoring system 250 can be implemented in a retail store. Food products can be disposed in shelving units. For example, egg cartons can be disposed in refrigerated shelving units. Arrays of sensors can be disposed above the shelves upon which the egg cartons. Each moisture sensor can detect moisture dissipated by a subset of egg cartons within a range of each sensor. An egg in an egg carton can break causing the egg yolk to leak into the carton releasing moisture to the environment. A moisture sensor within range of the of the egg carton can detect the increase in moisture caused by the egg yolk leaking into the egg carton. The moisture sensor can transmit the increase in moisture and the location of the sensor to the computing system 200.

The computing system 200 can determine the level of moisture in the location of the sensor based on the output of the moisture sensor The computing system 200 can compare the increased moisture level to a threshold moisture level. In response to determining the increased moisture level is greater than the threshold moisture level, the computing system 200 can determine at least one egg is damaged (e.g., cracked) within the subset of egg cartons in the range of the moisture sensor. The computing system 200 can transmit an alert including an estimated location of the cracked egg based on a location of the moisture sensor that sensed the increased moisture level.

In some embodiments, the computing system 200 can dynamically update the baseline moisture level and/or threshold value based on a change in moisture detected by two or more of the moisture sensors in the shelving unit. For example, egg cartons can be disposed in a refrigerated environment enclosed with by a door. For embodiments in which the shelving units are refrigerated units, an operation of the refrigeration units can insert or remove moisture causing the moisture sensors to detect increased or decreased moisture, respectively. Furthermore, each time a door of the refrigerated unit is opened increased moisture can be introduce into the shelving unit or moisture can escape the refrigerated unit causing a change in moisture detected by multiple sensors. The computing system 200 can determine a change in the environmental parameters based on the change in moisture detected by multiple sensors, and can update the baseline moisture level and/or threshold based on the environmental conditions collectively detected by the moisture sensors and/or by one or more other sensors, such as temperature sensors. The computing system 200 can determine that a uniform change was detected by the moisture sensors based on their proximity to a source of the environmental conditions causing the change in moisture levels. For example, the moisture sensors closest to the source of the environmental conditions causing the change in moisture levels can detect a larger change in environmental conditions as compared to the moisture sensors farther away from the environmental conditions. That is a subset of moisture sensors closest to the source (e.g., within a first radius) can detect a first uniform change in the moisture level, and a second subset of moisture sensors within a second radius (that is greater than the first radius) can detect a second uniform change in the moisture level.

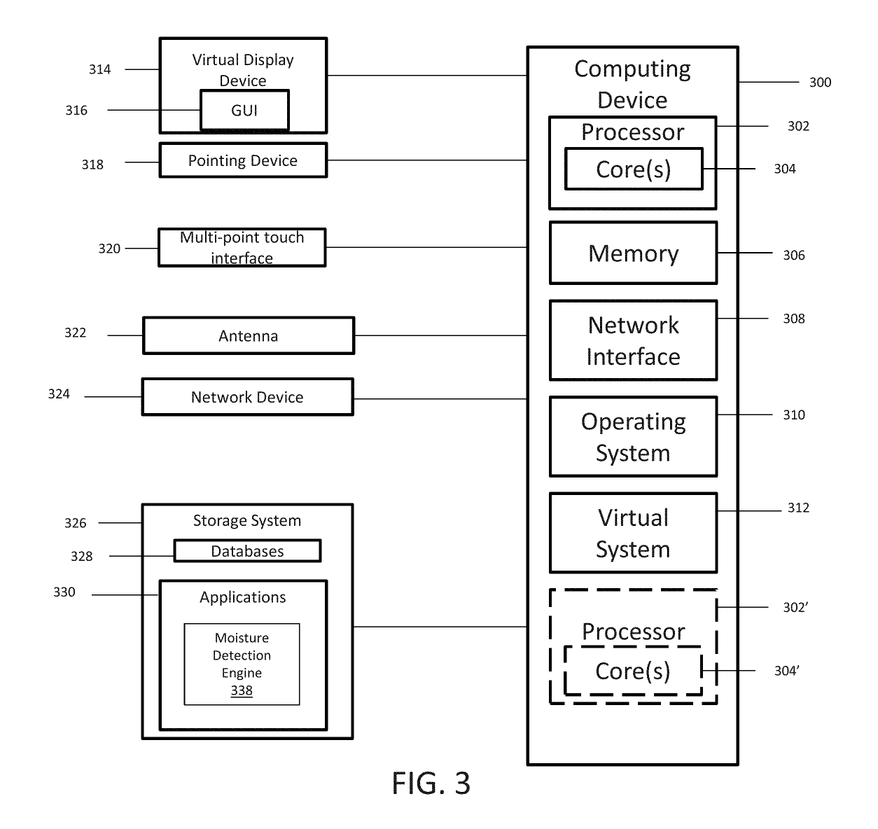

FIG. 3 is a block diagram of an example computing device for implementing exemplary embodiments of the present disclosure. Embodiments of the computing device 300 can implement embodiments of the moisture detection engine 338. The computing device 300 includes one or more non-transitory computer-readable media for storing one or more computer-executable instructions or software for implementing exemplary embodiments. The non-transitory computer-readable media may include, but are not limited to, one or more types of hardware memory, non-transitory tangible media (for example, one or more magnetic storage disks, one or more optical disks, one or more flash drives, one or more solid state disks), and the like. For example, memory 306 included in the computing device 300 may store computer-readable and computer-executable instructions or software (e.g., applications 330 including the moisture detection engine 338) for implementing exemplary operations of the computing device 300. The computing device 300 also includes configurable and/or programmable processor 302 and associated core(s) 304, and optionally, one or more additional configurable and/or programmable processor(s) 302′ and associated core(s) 304′ (for example, in the case of computer systems having multiple processors/cores), for executing computer-readable and computer-executable instructions or software stored in the memory 306 and other programs for implementing exemplary embodiments of the present disclosure. Processor 302 and processor(s) 302′ may each be a single core processor or multiple core (304 and 304′) processor. Either or both of processor 302 and processor(s) 302′ may be configured to execute one or more of the instructions described in connection with computing device 300.

Virtualization may be employed in the computing device 300 so that infrastructure and resources in the computing device 300 may be shared dynamically. A virtual machine 312 may be provided to handle a process running on multiple processors so that the process appears to be using only one computing resource rather than multiple computing resources. Multiple virtual machines may also be used with one processor.

Memory 306 may include a computer system memory or random access memory, such as DRAM, SRAM, EDO RAM, and the like. Memory 406 may include other types of memory as well, or combinations thereof.

A user may interact with the computing device 300 through a visual display device 314, such as a computer monitor, which may display one or more graphical user interfaces 316, multi touch interface 320 and a pointing device 318.

The computing device 300 may also include one or more storage devices 326, such as a hard-drive, CD-ROM, or other computer readable media, for storing data and computer-readable instructions and/or software that implement exemplary embodiments of the present disclosure (e.g., applications 330). Exemplary storage device 326 can also include one or more databases 328 for storing information regarding the physical objects. The databases 328 may be updated manually or automatically at any suitable time to add, delete, and/or update one or more data items in the databases. The databases 328 can include information such as environmental parameters database 230.

The computing device 300 can include a network interface 308 configured to interface via one or more network devices 324 with one or more networks, for example, Local Area Network (LAN), Wide Area Network (WAN) or the Internet through a variety of connections including, but not limited to, standard telephone lines, LAN or WAN links (for example, 802.11, T1, T3, 56 kb, X.25), broadband connections (for example, ISDN, Frame Relay, ATM), wireless connections, controller area network (CAN), or some combination of any or all of the above. In exemplary embodiments, the computing system can include one or more antennas 322 to facilitate wireless communication (e.g., via the network interface) between the computing device 300 and a network and/or between the computing device 300 and other computing devices. The network interface 308 may include a built-in network adapter, network interface card, PCMCIA network card, card bus network adapter, wireless network adapter, USB network adapter, modem or any other device suitable for interfacing the computing device 300 to any type of network capable of communication and performing the operations described herein.

The computing device 300 may run any operating system 310, such as any of the versions of the Microsoft® Windows® operating systems, the different releases of the Unix and Linux operating systems, any version of the MacOS® for Macintosh computers, any embedded operating system, any real-time operating system, any open source operating system, any proprietary operating system, or any other operating system capable of running on the computing device 300 and performing the operations described herein. In exemplary embodiments, the operating system 310 may be run in native mode or emulated mode. In an exemplary embodiment, the operating system 310 may be run on one or more cloud machine instances.

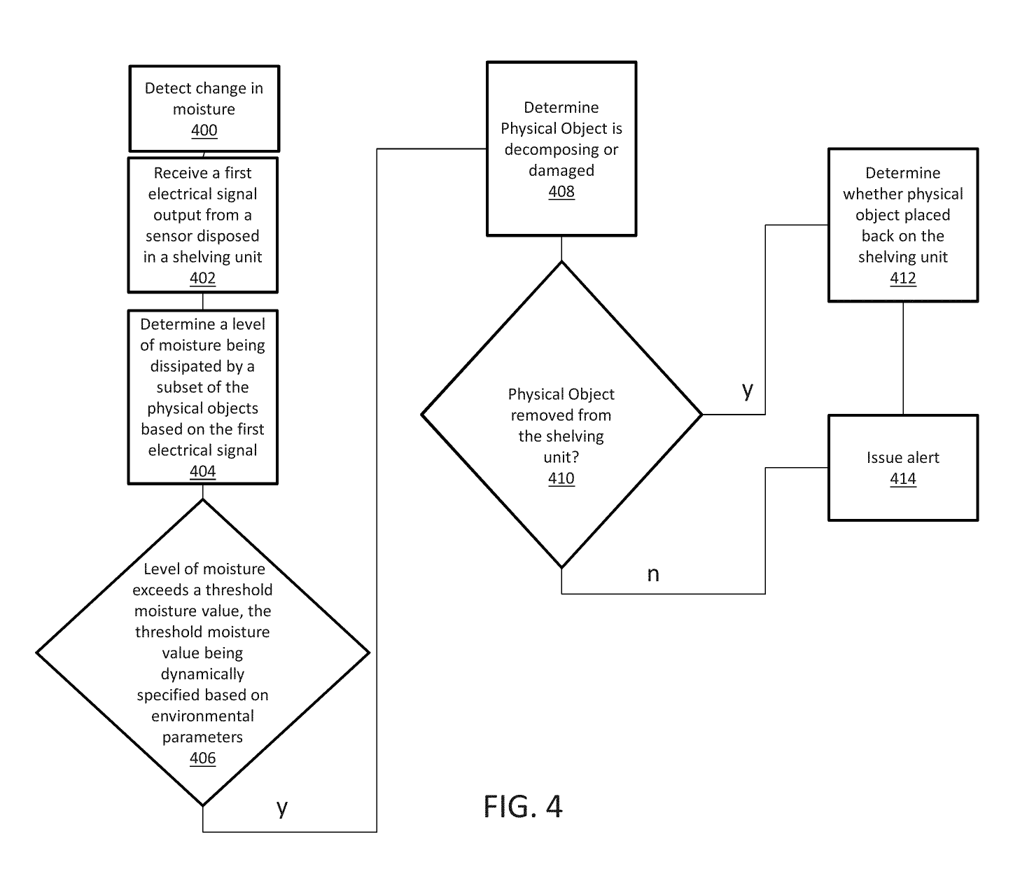

FIG. 4 is a flowchart illustrating a perishable food monitoring process according to exemplary embodiments of the present disclosure. In operation 400, moisture sensors including a first moisture sensor (e.g. moisture sensor 110a shown in FIG. 1B) configured to detect moisture can be disposed in a shelving unit (e.g. shelving unit shown in FIG. 1B 114) with respect to physical objects (e.g. physical objects 112a-c shown in FIG. 1B) supported by the shelving unit. The first moisture sensor can detect moisture within a range (e.g. range 114a shown in FIG. 1B). The range can include a set of physical objects disposed within the range. The physical objects can be edible items and can dissipate moisture when decomposing or damaged. The first moisture sensor can encode an amount of moisture detected by the first moisture sensor in an electrical signal and transmit the electrical signal to a computing system (e.g. computing system 200 shown in FIG. 2).

In operation 402, the computing system can receive the electrical signal from the first moisture sensor. The computing system can determine the level of moisture detected from the first moisture sensor based on the electrical signal. The computing system can also determine the location of the moisture sensor. In operation 404, the computing system can query the environmental parameters database (e.g. environmental parameters database 230 as shown in FIG. 2) to retrieve the environmental parameters causing moisture in the location of the first sensor. The environmental database stores sensed or measured environmental parameters of the shelving units and/or the environment surrounding the shelving units. The environment parameters can impact the level of moisture in around the shelving units. The environmental parameter can be but are not limited to, increased and decreased humidity, increased and decreased temperature, condensation, liquid contained within the physical objects and other environmental parameters causing moisture. Based on the retrieved environmental parameters the computing system can eliminate the amount of moisture caused by the environmental parameters from the amount of moisture detected by the first moisture sensor and the computing system can determine remaining amount of moisture can be dissipated by at least one physical object within the range of the first moisture sensor.

In operation 406, the computing system can determine whether the level of moisture dissipated by at least one physical object is greater than a predetermined threshold level based on the environmental parameters. In some embodiments, the computing system can receive electrical signals from multiple moisture sensors. The computing system can determine a change in the environmental parameters and can update the environmental parameters database. The computing system can also dynamically update the predetermined threshold. For example, the computing system can use the current moisture sensed by each of the moisture sensors and/or one or more stored environmental parameters to set the threshold value. The computing system can to compare the moisture level determined based on the first electrical signal and the threshold moisture level of the location of the first sensor and determine whether the moisture level determined based on the first electrical signal exceeds the threshold moisture level of the location of the first moisture sensor. In operation 408, in response to determining the moisture level is above the threshold level the computing system can determine at least one physical object within the range of the first moisture sensor is decomposing or damaged. The computing system can determine the location of the damaged or decomposing physical object based on the location of the first sensor.

In operation 410, the computing system can receive a second electrical signal from the first sensor. The computing system can determine the moisture level detected by the first moisture sensor based on second electrical signal. In response to determining the moisture level determined based on the second electrical has decreased as compared to moisture level determined based on the first electrical signal, the computing system can determine the damaged or decomposing has been removed from the shelving unit. In some embodiments, the computing system can determine the moisture level determined based on the second electrical signal has not decreased and the computing system can determine the damaged or decomposing has not been removed from the shelving unit. The computing system can issue an alert (operation 414).

In operation 412, the computing system can receive a third electrical signal from the first moisture sensor. The computing system can determine the moisture level detected by the first moisture sensor based on third electrical signal. In response to determining the moisture level determined based on the third electrical is greater than the moisture level determined based on the second electrical signal, the computing system can determine the damaged or decomposing has been placed back on the shelving unit.

In operation 414, the computing system can issue an alert with respect to the status of the damaged or decomposing physical object. The alert can include the location of the damaged or decomposing physical object.

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes a plurality of system elements, device components or method steps, those elements, components or steps may be replaced with a single element, component or step. Likewise, a single element, component or step may be replaced with a plurality of elements, components or steps that serve the same purpose. Moreover, while exemplary embodiments have been shown and described with references to particular embodiments thereof, those of ordinary skill in the art will understand that various substitutions and alterations in form and detail may be made therein without departing from the scope of the present disclosure. Further still, other aspects, functions and advantages are also within the scope of the present disclosure.

Exemplary flowcharts are provided herein for illustrative purposes and are non-limiting examples of methods. One of ordinary skill in the art will recognize that exemplary methods may include more or fewer steps than those illustrated in the exemplary flowcharts, and that the steps in the exemplary flowcharts may be performed in a different order than the order shown in the illustrative flowcharts.

We claim:

1. A perishable food monitoring system, the system comprising:
a shelving unit including a support surface for supporting physical objects;
a first set of moisture sensors disposed above and spaced away from the support surface, the first set of moisture sensors configured to detect moisture being dissipated by the physical objects supported by the support surface and to output electrical signals corresponding to detected moisture being dissipated by the physical objects; and
a computing system operatively coupled to the first set of sensors, the computing system being programmed to:
receive a first electrical signal from at least one sensor included in the first set of sensors;
determine a level of moisture being dissipated by at least a subset of the physical objects based on the first electrical signal;
determine whether at least one of the physical objects in the subset is damaged or decomposing in response to a determination that the level of moisture exceeds a threshold moisture value, the threshold moisture value being dynamically specified based on at least one environmental parameter;
estimate a location of the at least one physical object in response to determining the level of moisture exceeds the threshold moisture value and determining a first sensor location for the at least one sensor; and
transmitting an alert in response determining the location of the at least one physical object.

2. The system in claim 1, wherein the computing system is further programmed to:
determine the level of moisture being dissipated by the at least subset of the physical objects decreases based on the first electrical signal received from the at least one sensor; and
determine the at least one of the physical object in the subset which is determined to be damaged or decomposing is removed from the shelving unit in response to the decrease.

3. The system in claim 2, wherein the computing system is further programmed to:
determine the level of moisture being dissipated by at least the subset of the physical objects increases subsequent to the decrease based on the first electrical signal received from the at least one sensor; and
determine the at least one of the physical objects in the subset which is determined to be damaged or decomposing is placed back on the shelving unit.

4. The system in claim 1, wherein the shelving unit is refrigerated.

5. The system in claim 1, wherein the at least one environmental parameter is humidity.

6. The system in claim 5, wherein the computing system dynamically changes the threshold moisture value based on a change detected in the environmental parameter by at least two sensors in the first set of sensors and in response to not detecting at least one of the physical objects in the subset is damaged or decomposing.

7. The system of claim 1, wherein the first set of sensors forms an array that is disposed parallel to the support surface, and the system further comprising:
a second set of sensors disposed in an array perpendicular to the support surface.

8. The system in claim 7, wherein the computing system is programmed to:
receive a second electrical signal from at least one sensor in the second set of sensors; and
determine whether the at least one of the physical objects in the subset is damaged or decomposing based on the second electrical signal.

9. The system in claim 8, wherein the computing system is programmed to estimate the location of the at least one physical object producing excess moisture by determining the first sensor location of the at least one sensor from the first set of sensors and a second sensor location of the at least one sensor from the second set of sensors.

10. The system in claim 1, wherein the physical objects are containers housing a plurality of edible items.

11. A perishable food monitoring method, the system comprising:
receiving, by a computing system, a first electrical signal output from at least one sensor included in a first set of sensors disposed with respect to a support shelf in an shelving unit;
determining, via the computing system, a level of moisture being dissipated by at least a subset of the physical objects based on the first electrical signal;
determining, via the computing system, whether at least one of the physical objects in the subset is damaged or decomposing in response to a determination that the level of moisture exceeds a threshold moisture value, the threshold moisture value being dynamically specified based on at least one environmental parameter;
estimating, via the computing system, a location of the at least one physical object producing excess moisture in response to determining the level of moisture exceeds the threshold moisture value and determining a location of the at least one sensor; and
in response to determining the location of the at least one physical object producing, transmitting an alert;

12. The method in claim 11, further comprising:
determining, via the computing system, the level of moisture being dissipated by the at least subset of the physical objects decreases based on the first electrical signal received from the at least one sensor; and
determining, via the computing system, the at least one of the physical object in the subset which is determined to be damaged or decomposing is removed from the shelving unit in response to the decrease.

13. The method in claim 12, further comprising:
determining, via the computing system, the level of moisture being dissipated by at least the subset of the physical objects increases subsequent to the decrease based on the first electrical signal received from the at least one sensor; and
determining, via the computing system, the at least one of the physical objects in the subset which is determined to be damaged or decomposing is placed back on the shelving unit.

14. The method in claim 11, wherein the shelving unit is refrigerated.

15. The method in claim 11, wherein the at least one environmental parameter is humidity.

16. The method in claim 15, further comprising, dynamically changing, via the computing system, the threshold moisture value based on a change detected in the environmental parameter by at least two sensors in the first set of sensors and in response to not detecting at least one of the physical objects in the subset is damaged or decomposing.

17. The method of claim 11, wherein the first set of sensors forms an array that is disposed parallel to the support surface; and
a second set of sensors disposed in an array perpendicular to the support surface.

18. The method in claim 17, further comprising:
receiving, via the computing system, a second electrical signal from at least one sensor in the second set of sensors; and
determining, via the computing system, whether the at least one of the physical objects in the subset is damaged or decomposing based on the second electrical signal.

19. The method in claim 18, further comprising estimating, via the computing system, the location of the at least one physical object producing excess moisture by determining the first sensor location of the at least one sensor from the first set of sensors and a second sensor location of the at least one sensor from the second set of sensors.

20. The method in claim 11, wherein the physical objects are containers housing a plurality of edible items.
